(19) United States
(12) Patent Application Publication
(10) Pub. No.: US 2025/0302320 A1
(43) Pub. Date: Oct. 2, 2025

(54) DEEP EQUILIBRIUM MODEL BASED SYSTEMS AND METHODS FOR ESTIMATING VITAL SIGNS

(71) Applicant: Mitsubishi Electric Research Laboratories, Inc., Cambridge, MA (US)

(72) Inventors: Suhas Lohit, Cambridge, MA (US); Vineet Shenoy, Princeton, NJ (US); Hassan Mansour, Newton, MA (US); Tim Marks, Newton, MA (US)

(73) Assignee: Mitsubishi Electric Research Laboratories, Inc., Cambridge, MA (US)

(21) Appl. No.: 18/619,583

(22) Filed: Mar. 28, 2024

(51) Int. Cl. A61B 5/024; A61B 5/00; G06N 3/08

(52) U.S. Cl. CPC A61B 5/02427; A61B 5/7203; A61B 5/7264; A61B 5/7267; G06N 3/08

(57) ABSTRACT

A remote photoplethysmography system for estimating a vital sign signal of a subject comprises circuitry configured to collect a sequence of images of different regions of skin of the subject. Each region includes pixels of different intensities indicative of variation of coloration of the skin. The sequence of images are transformed into a sequence of imaging photoplethysmography (iPPG) signals indicative of variation of the vital signs of the subject in time domain. The iPPG signals are subject to structured non-Gaussian noise. The iPPG signals are denoised by solving a structured recovery problem with a regularizer enforcing a neural network discovered structure on the iPPG signals. The regularizer includes a learned regularization term implemented using a deep equilibrium model. The processor vital sign signal corresponding to the denoised iPPG signals is output via an interface.

### TECHNICAL FIELD

The present disclosure relates generally to remotely monitoring vital signs of subjects and more particularly to imaging photoplethysmography (iPPG) systems and methods for remote measurement of vital signs.

### BACKGROUND

Vital signs of a person, for example heart rate (HR), heart rate variability (HRV), respiration rate (RR), or blood oxygen saturation, serve as indicators of a person's current state and as a potential predictor of serious medical events. For this reason, vital signs are extensively monitored in inpatient and outpatient care settings, at home, and in other health, leisure, and fitness settings. One way of measuring the vital signs is plethysmography which corresponds to measurement of volume changes of an organ or a body part of a person. There are various implementations of plethysmography, such as Photoplethysmography (PPG) which is an optical measurement technique that evaluates a time-variant change of light reflectance or transmission of an area or volume of interest, which can be used to detect blood volume changes in microvascular bed of tissue. PPG is based on a principle that blood absorbs and reflects light differently than surrounding tissue, so variations in the blood volume with every heartbeat affect light transmission or reflectance correspondingly.

Conventional non-invasive instruments for measuring vital signs of a person, often need to be attached to the skin of the person, for instance to a fingertip, earlobe, or forehead. This may not be pleasant to the person for several reasons. Additionally, the sensor's incidence window may be too large or too small for some patients and as such may not provide correct readings. Furthermore, in view of outbreaks of contagious diseases such as the SARS-CoV-2 based novel coronavirus disease, the use of non-contact non-invasive techniques for measuring vital signs has become essential. Recent years have witnessed increasing interest in non-contact monitoring of vital signs using cameras, particularly for telemedicine, including estimation of heart rate, breathing rate, and blood pressure from video of the face or some other body part of a subject. The main advantage of monitoring vital signs of a person using a camera, rather than using a conventional contact sensor is easier usage. Cameras also provide vital sign information over a larger spatial region naturally, compared to having a highly localized contact sensor. Also the granularity of the output data can be fine tuned based on the resolution and capability of the camera sensors.

In addition to healthcare, remote monitoring can be used in safety-critical applications such as driving or heavy equipment operation, as there is no requirement of attaching a contact sensor to the operator's body, which can otherwise hinder normal operation of the users. Cameras recording facial videos capture the subtle changes in skin color corresponding to the blood volume pulse. However, the captured videos are also marred by noise due to several factors. For example, some vital signs such as the blood volume pulse signal component is a small fraction of the pixel intensity and can be easily masked by illumination changes and motion. Therefore, in order to perform an accurate measurement of the vital signs, it is important various types of noises are taken into consideration as a part of the vital sign estimation.

Some attempts in this direction have been made using blind source separation methods, model based methods, and data driven methods. However, these approaches have not been effective in recovering or extracting the underlying pulse signal due to several reasons. For example, the model-based methods are not exact and cannot capture all variations in real-world data. Also, they are not effective in recovering the underlying pulse signal as the handcrafted constraints are too simple and do not account for all the characteristics of the signal. On the other hand, purely data-driven deep learning-based methods lack good interpretability of the underlying approach since they are black-box methods and offer low interpretability. Another challenge faced by conventional vital sign estimation approaches is that the separation between pulse signal and noise is sub-optimal and does not meet the quality metrics of data required for many real world applications.

Hence there is a need for developing solutions for remote estimation of vital signs, that are effective, are based on data-driven modeling of both the pulse wave as well as the structured noise, and at the same time maintain interpretability. Furthermore, there is also a need to develop solutions that can effectively separate noise from the useful signal to reconstruct the underlying pulse signal from the input data.

### SUMMARY

Accordingly, it is an objective of some embodiments to estimate vital signs of a subject with high accuracy. It is also an objective of some example embodiments to provide accurate measurements of vital signs of a subject located in a volatile environment in which several unique sources of noise exist. Some example embodiments provide such solutions with good interpretability of the underlying approach while providing accurate measurements of the vital signs. Some example embodiments are also directed towards the objective of providing such solutions that take into consideration all the characteristics of the measured signals when attempting to recover the underlying pulse signals from the measured signals. In this regard, some embodiments deploy neural networks that learn characteristics in the form of signal priors of the measured signals such that these characteristics are utilized for separating the noise from the signal.

Some embodiments are based on a realization that the signal extracted from the input data is the sum of the pulse signal and noise, and the Fourier coefficients of the pulse signal and the noise signal can be sparse. Armed with this realization, a sparse optimization problem may be solved for the frequency coefficients and noise using proximal gradient descent approach. However, such a realization assumes the Fourier coefficients of the pulse signal and the noise signal to be sparse. Some embodiments aim to relax these sparsity assumptions by incorporating a learnable prior. Also, since deep learning methods show significant performance improvements over model based approaches, some embodiments realize the learnable prior through deep learning networks that accept primary colour light frames directly as input.

Some embodiments also recognize that achieving imaging photoplethysmography (iPPG) or remote photoplethysmography (rPPG) performance gains should integrate the theoretical strengths of inverse problem formulation methods with the empirical benefits of deep learning methods. In this regard, approaches based on unrolling optimization algorithms integrate learnable parameters into traditional iterative algorithms, harnessing the power of learning while exploiting known structures and retaining interpretability. Particularly, the unrolling based approach imposes a learned prior on the signal and noise instead of sparsity priors, and solves for the corresponding proximal operators by “unrolling” proximal gradient descent for T iterations, where T is a hyperparameter determined empirically. These algorithms repeatedly apply two steps: first, they ensure that the intended result is consistent with measurements by minimizing a data fidelity term using a learned or fixed forward operator, and second, they apply a signal denoiser using a learned signal prior to fit the solution to a ground truth signal.

However, some embodiments also realize that it may be sub-optimal to apply only a single denoising step in each of the unrolled iterations. Instead, further denoising may be achieved by repeatedly applying the denoising operator until the output converges. Expanding upon these understandings, some embodiments consider that the frequency coefficients of the underlying heart rate signal can be obtained as a fixed point of the denoising operator. As a result of meticulous experimentations, it is a recognition of several embodiments that Deep Equilibrium Models (also referred to as DEQs) solve a non-linear system prescribed by a criterion such as the endpoint of an ODE flow or the root of an equation, and backpropagate through this point. Thus, DEQs decouple the choice of solver from the computation of the endpoint or root, and can backpropagate through this point analytically and independently of the forward pass. Accordingly, for estimating vital signs of a subject, some example embodiments are directed towards providing an unrolling gradient descent based framework with a deep equilibrium model (DEQ) as the denoiser. Some embodiments are also directed towards finding a deep equilibrium model for the overall proximal gradient descent iterations of the unrolling based framework.

It is a realization of some embodiments that conventional deep learning models learn by applying explicit functions to inputs, building a computation graph along the way, and back-propagating through this graph to update parameters after a loss computation. It is also a realization of some embodiments that implicit models such as Deep Equilibrium Models do not build computation graphs; instead, such models solve a non-linear system prescribed by a criterion such as the endpoint of an ODE flow or the root of an equation, and backpropagate through this point. Thus, some embodiments recognized that implicit models such as DEQs decouple the choice of solver from the computation of the endpoint or root, and can backpropagate through this point analytically and independently of the forward pass. Using this approach, training and prediction in these networks require only constant memory, regardless of the effective “depth” of the network.

Some example embodiments leverage these properties of DEQs by using them as a pseudo proximal operator for the purpose of estimating vital signs of a subject. Some embodiments are based on an observation that the frequencies of the iPPG signals in various RPPG applications converge towards some fixed equilibrium points. Armed with this observation, some embodiments are based on recognizing that the structure of the iPPG signals can be learned and/or represented as a deep equilibrium model that directly finds these equilibrium points via root-finding techniques.

As a result, the iPPG signals can be denoised by solving a structured recovery problem with a regularizer enforcing a DEQ-based neural network discovered structure on the iPPG signals. However, several example embodiments recognize that instead of an explicit regularization term such as an l₁ norm or total variation prior, the signal structure can be learned rather than solved directly. This, in turn, improves the accuracy of the vital sign estimation.

Some embodiments are based on recognizing that the DEQ offers additional flexibility for integration with a structured recovery solver. Deep equilibrium models (DEQs) are a class of neural network architectures that seek to directly solve for the equilibrium of a system rather than iteratively approximating it through layers of computation. As a result, the DEQs can be applied not only within the framework of the structured recovery but also as a standalone operator. Notably, when the DEQs are applied as a standalone operator, the DEQs do not suffer from the limitations of other neural networks that are trained to mimic the number of iterations of the structured recovery solver. Instead for each execution, the DEQs are executed as many times as necessary to find the equilibrium point of the input.

Towards these ends, it is an objective of some example embodiments to provide systems, methods and computer program products that effectively estimate vital signs of a subject using a DEQ-based unrolling optimization approach. The disclosed embodiments model the signal extracted from video of a subject as the sum of an underlying pulse signal and noise. However, instead of explicitly imposing a handcrafted prior (e.g., sparsity in the frequency domain) on the signal, some example embodiments learn priors on the signal and noise using neural networks. Some of the disclosed embodiments solve for the underlying pulse signal by unrolling proximal gradient descent, wherein the algorithm alternates between gradient descent steps and application of learned denoisers, which replace handcrafted priors and their proximal operators. In other words, some embodiments combine a model-based approach with a data-driven deep neural network for estimation of the vital signs.

Accordingly, some example embodiments provide a remote photoplethysmography (RPPG) system for estimating a vital sign signal of a subject. The system comprises memory configured to store instructions and a processor configured to execute the instructions to cause the RPPG system to collect a sequence of images of different regions of skin of the subject, each region including pixels of different intensities indicative of variation of coloration of the skin. The processor is also configured to transform the sequence of images into a sequence of imaging photoplethysmography (iPPG) signals indicative of variation of the vital signs of the subject in a time domain. The iPPG signals are subject to structured non-Gaussian noise. The processor is further configured to denoise the iPPG signals by solving a structured recovery problem with a regularizer enforcing a neural network discovered structure on the iPPG signals. The regularizer includes a learned regularization term implemented using a deep equilibrium model (DEQ). The processor outputs the vital sign signal corresponding to the denoised iPPG signals via an interface.

In yet another example embodiment, a computer-implemented method for estimating a vital sign signal of a subject is provided. The method comprises collecting a sequence of images of different regions of skin of the subject, each region including pixels of different intensities indicative of variation of coloration of the skin. The method also comprises transforming the sequence of images into a sequence of imaging photoplethysmography (iPPG) signals indicative of variation of the vital signs of the subject in a time domain. The iPPG signals are subject to structured non-Gaussian noise. The method further comprises denoising the iPPG signals by solving a structured recovery problem with a regularizer enforcing a neural network discovered structure on the iPPG signals. The regularizer includes a learned regularization term implemented using a deep equilibrium model (DEQ). The vital sign signal corresponding to the denoised iPPG signals is then output via an interface.

In yet some other example embodiments, a non-transitory computer readable medium having stored thereon computer executable instructions for performing a method for estimating the vital sign signal of the subject is provided. The method comprises collecting a sequence of images of different regions of skin of the subject, each region including pixels of different intensities indicative of variation of coloration of the skin. The method also comprises transforming the sequence of images into a sequence of imaging photoplethysmography (iPPG) signals indicative of variation of the vital signs of the subject in a time domain. The iPPG signals are subject to structured non-Gaussian noise. The method further comprises denoising the iPPG signals by solving a structured recovery problem with a regularizer enforcing a neural network discovered structure on the iPPG signals. The regularizer includes a learned regularization term implemented using a deep equilibrium model (DEQ). The vital sign signal corresponding to the denoised iPPG signals is then output via an interface.

### BRIEF DESCRIPTION OF THE DRAWINGS

The presently disclosed embodiments will be further explained with reference to the following drawings. The drawings shown are not necessarily to scale, with emphasis instead generally being placed upon illustrating the principles of the presently disclosed embodiments.

FIG. 1A illustrates a flowchart of a method for estimating a vital sign of a subject, according to some example embodiments;

FIG. 1B illustrates a workflow for the method for estimating a vital sign of a subject, according to some example embodiments;

FIG. 1C illustrates a flowchart of a method for extracting multi-dimensional time series data from a video of the subject, according to some example embodiments;

FIG. 2A illustrates a block diagram of an unrolled Deep Equilibrium Model-based imaging photoplethysmography (UDEQ-iPPG) system for estimating a vital sign of a subject, according to some example embodiments;

FIG. 2B illustrates a flowchart of some steps performed by the UDEQ-iPPG system of FIG. 2A, according to some example embodiments;

FIG. 2C illustrates two generic iterations of a UDEQ-iPPG algorithm executed by the system of FIG. 2A, according to some example embodiments;

FIG. 2D illustrates a block diagram of a DEQ operator utilized in each iteration of the UDEQ-iPPG algorithm of FIG. 2C, according to some example embodiments;

FIG. 3A illustrates a block diagram of a Deep Equilibrium Proximal Gradient Descent (DE-Prox) based imaging photoplethysmography (DE-Prox-iPPG) system for estimating a vital sign of a subject, according to some example embodiments;

FIG. 3B illustrates a flowchart of some steps performed by the DE-Prox-iPPG system of FIG. 3A, according to some example embodiments;

FIG. 3C illustrates an iterative DE-Prox-iPPG algorithm executed by the system of FIG. 3A, according to some example embodiments;

FIG. 3D illustrates an architecture of denoisers utilized by the DE-Prox-iPPG system of FIG. 3A, according to some example embodiments;

FIG. 4 illustrates a block diagram of a computing system for impementing some components of an iPPG system, according to some example embodiments;

FIG. 5 illustrates a patient monitoring system using the iPPG system of FIG. 4, according to some example embodiments; and

FIG. 6 illustrates a vehicle assistance system using the iPPG system of FIG. 4, according to some example embodiments.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. Contemplated are various changes that may be made in the function and arrangement of elements without departing from the spirit and scope of the subject matter disclosed as set forth in the appended claims.

Specific details are given in the following description to provide a thorough understanding of the embodiments. However, understood by one of ordinary skill in the art can be that the embodiments may be practiced without these specific details. For example, systems, processes, and other elements in the subject matter disclosed may be shown as components in block diagram form in order not to obscure the embodiments in unnecessary detail. In other instances, well-known processes, structures, and techniques may be shown without unnecessary detail in order to avoid obscuring the embodiments. Further, like-reference numbers and designations in the various drawings may indicate like elements.

Also, individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed but may have additional steps not discussed or included in a figure. Furthermore, not all operations in any particularly described process may occur in all embodiments. A process may correspond to a method, a function, a procedure, a subroutine, a subprogram, etc. When a process corresponds to a function, the function's termination can correspond to a return of the function to the calling function or the main function.

Furthermore, embodiments of the subject matter disclosed may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine-readable medium. A processor(s) may perform the necessary tasks.

Contactless monitoring of vital signs such as heart rate is an important tool for improved quality of life and preventative healthcare. Recently, non-contact, remote PPG (RPPG) devices for unobtrusive measurements have been introduced. RPPG utilizes light sources or, in general, radiation sources disposed remotely from a subject of interest. Similarly, a detector, e.g., a camera or a photo detector, can be disposed remotely from the person of interest. Therefore, remote photoplethysmography systems and devices are considered unobtrusive and well suited for medical as well as non-medical everyday applications. One of the advantages of camera-based vital signs monitoring versus on-body sensors is the high ease-of-use: there is no need to attach a sensor; just aiming the camera at the person is sufficient. Another advantage of camera-based vital signs monitoring over on-body sensors is the potential for achieving motion robustness: cameras have greater spatial resolution than contact sensors, which mostly include a single-element detector. RPPG technology faces a major challenge when it comes to providing accurate measurements under motion/light distortions. Particularly, the pulse signal that contains information from the subject's body is engulfed with noise that gets into the measurement due to several reasons. As such, the vital sign component is only a small fraction of the pixel intensity and can be easily masked by illumination changes and motion. Therefore, in order to perform an accurate measurement of the vital signs, it is important various types of noises are taken into consideration as a part of the vital sign estimation.

However, explicit modelling of individual components of the measured signal is a cumbersome task. Also, fixed or handcrafted models cannot be applied to every situation. What is desired is a framework that models individual components of the measured signal using trainable parameters so that the vital sign estimation system can be fine-tuned according to needs. Example embodiments disclosed herein provide solutions for remote estimation of vital signs, that are effective, are based on data-driven modeling of both the pulse wave as well as the structured noise, and at the same time maintain interpretability. In this regard, some example embodiments provide deep equilibrium learning based approaches for imaging photoplethysmography (iPPG). Such approaches use deep learning methods set in an inverse problem framework that estimate the underlying pulse signal and vital sign such as heart rate from video using a learned proximal gradient descent algorithm. Towards this end, some embodiments formulate waveform recovery as an optimization problem in which the sum of a data fidelity term and a regularization term is to be minimized. Instead of an explicit regularization term such as an l₁ or total variation prior, some example embodiments define a learned regularization term whose operators are realized as operations through deep equilibrium models (DEQs). Some embodiments integrate the deep equilibrium models as a standalone pseudo proximal operator in unrolling techniques. Some other embodiments integrate the deep equilibrium models as the fixed-point iteration of proximal gradient descent.

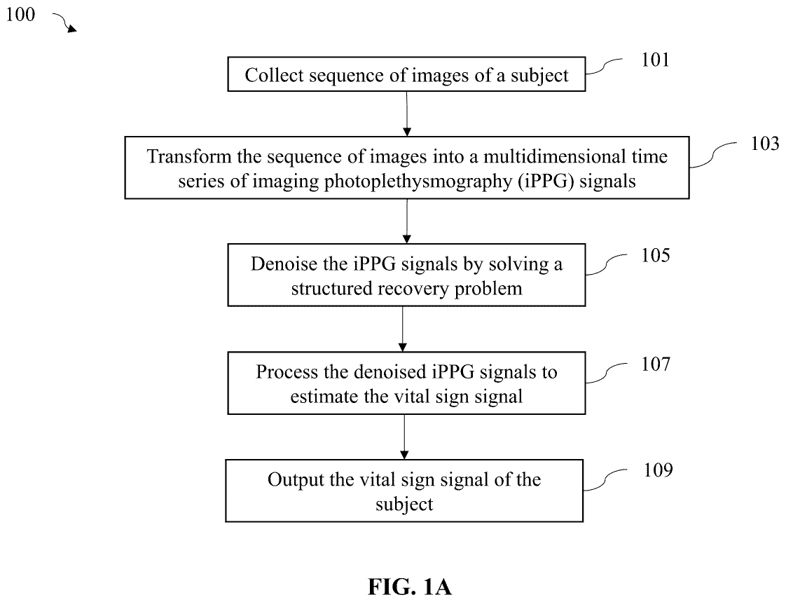

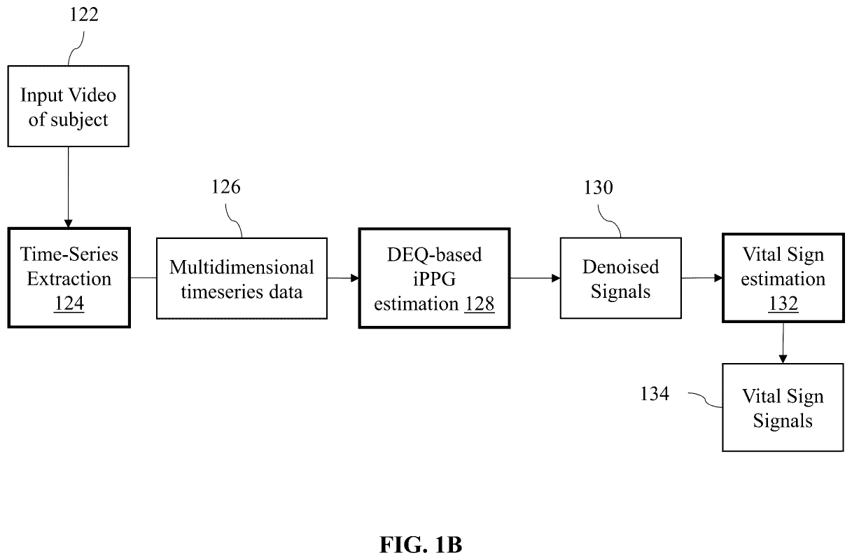

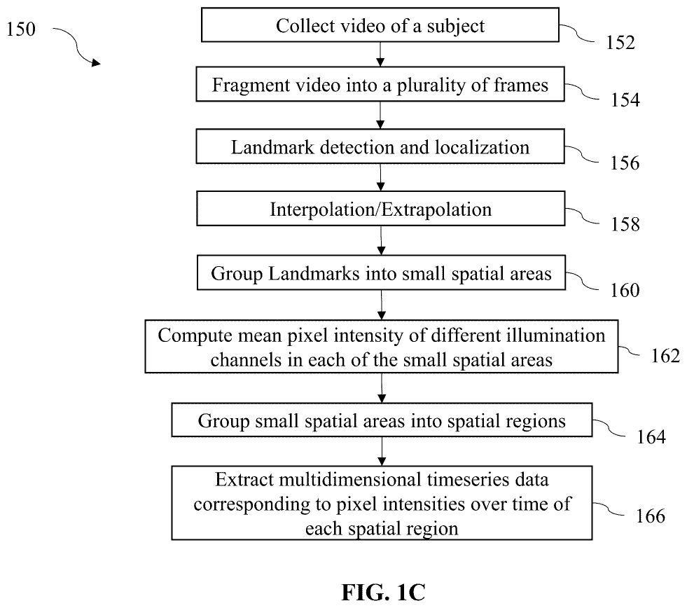

FIG. 1A illustrates a flowchart of a method 100 for estimating a vital sign of a subject, according to some example embodiments. The method 100 may be executed by an imaging photoplethysmography (iPPG) system that is realized in software or a combination of hardware and software. A sequence of images of a subject whose vital sign is to be estimated is collected 101. The subject may be a human or an animal. The sequence of images of the subject may correspond to a video of the subject, where the video may be a live video in real time or a pre-recorded video. The video may be captured under a suitable illumination spectrum such as in the NIR or visible or a broad spectrum visible and NIR wavelengths. The sequence of images capture at least one body part of the subject under consideration. For example, the sequence of images may capture the face of the subject in each of the images or at least some of the images in the sequence of images of the subject.

The method 100 further comprises transforming 103 the sequence of images of the subject into a multidimensional time series of imaging photoplethysmography (iPPG) signals. In this regard, each image of the sequence of images may be partitioned into a plurality of spatial regions and an iPPG signal may be determined corresponding to each spatial region. Thus, the time series data comprises a plurality of iPPG waveforms/signals indicative of variation of the vital signs of the subject in a time domain. Since the sequence of images are collected remotely from the subject, the iPPG signals may be subject to structured non-Gaussian noise. A detailed description of the steps leading to transformation of the sequence of images into time series iPPG signals is described later with reference to FIG. 1C.

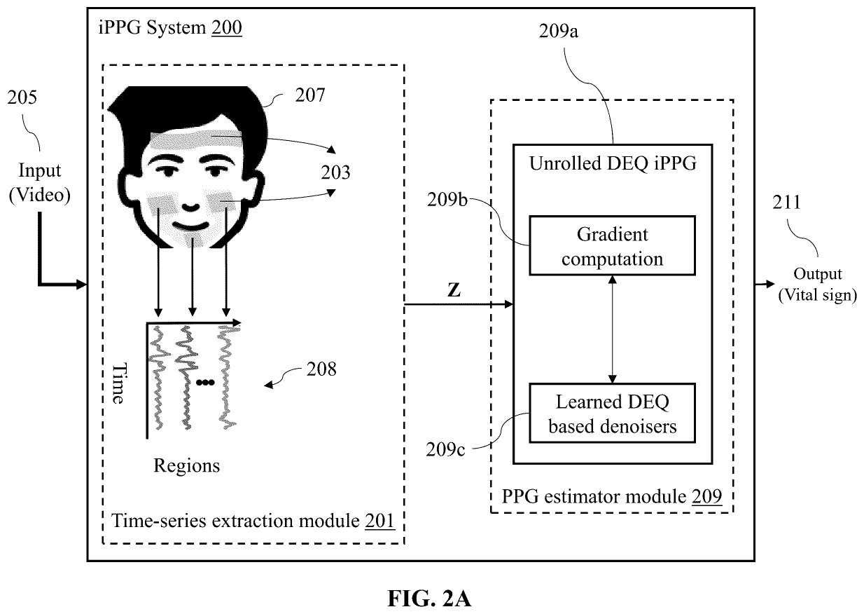

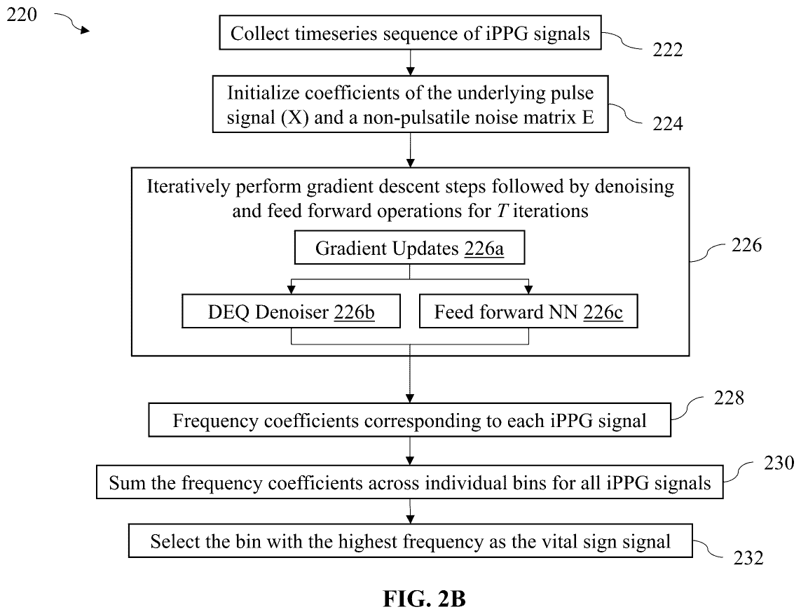

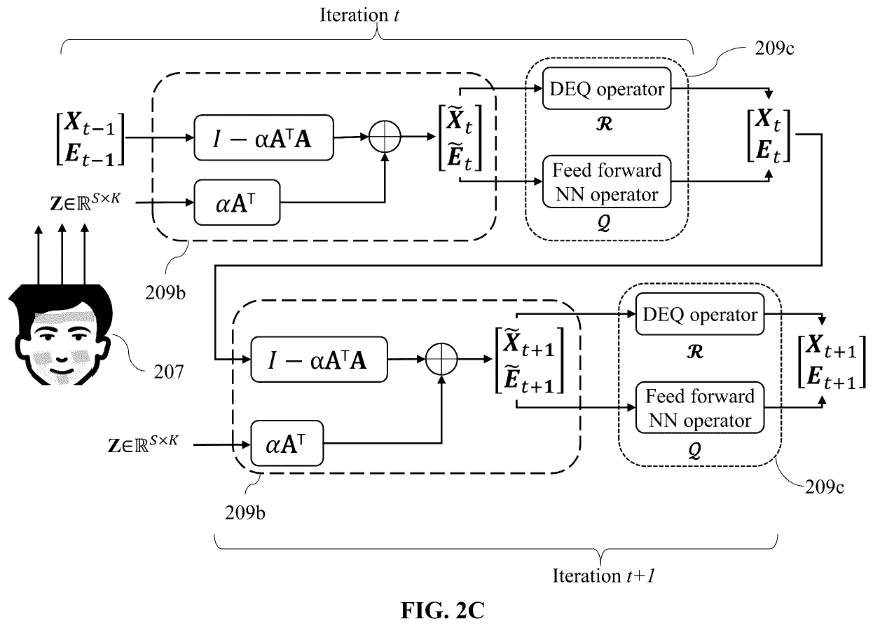

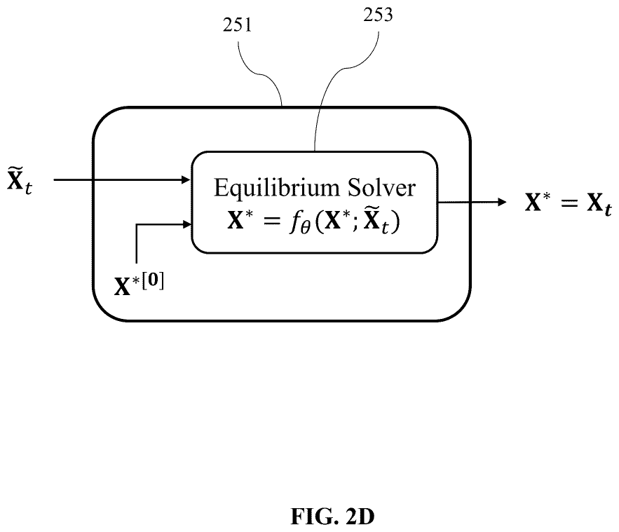

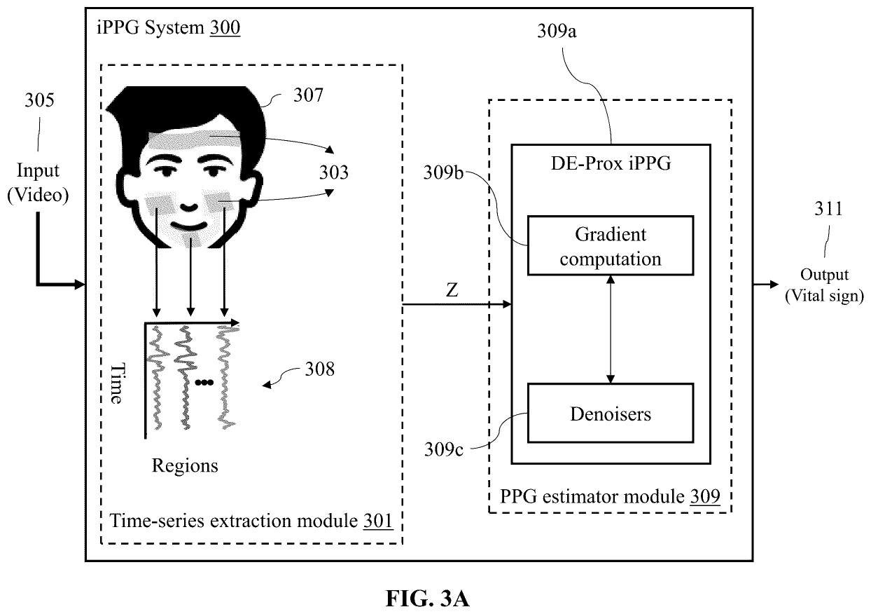

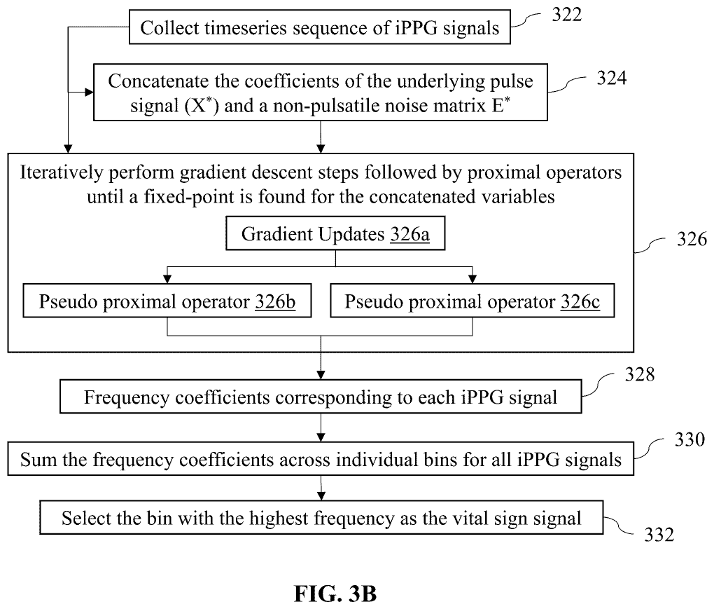

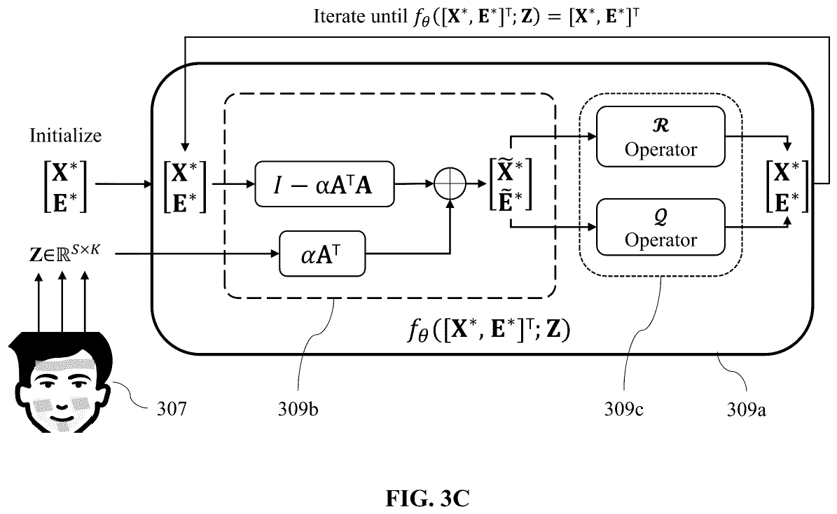

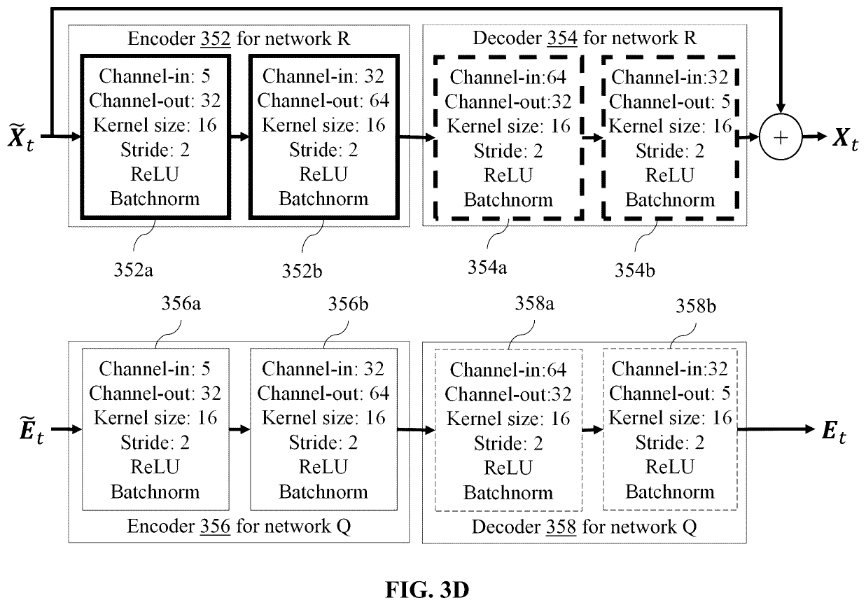

The iPPG signals may be denoised 105 to filter out the noise from the iPPG signal such that a substantial component of the denoised signal corresponds to the underlying pulsatile component. In this regard, denoising 105 may be performed by solving a structured signal recovery problem where a regularizer enforces a neural network discovered structure on the iPPG signals. According to some embodiments, the regularizer includes a learned regularization term implemented using a deep equilibrium model (DEQ). Details regarding the denoising of iPPG signals using DEQ will be described later with reference to FIGS. 2A-3D.

The denoising of the iPPG signals returns denoised signals that are processed 107 to estimate the vital sign signal of the subject. The process of determining the vital sign depends on the desired vital sign. For instance, if the desired vital sign is the heart rate, the Fourier transform of the denoised iPPG is first computed and the frequency at which the magnitude of the Fourier transform is highest is treated as the estimated heart rate. If the desired vital sign is the heart rate variability, peak locations in the denoised iPPG signal are determined and the time duration between the successive peaks becomes the estimate for the heart rate variability. The estimated vital sign signal may then be output 109 via an interface. For example, the estimated vital sign signal may be displayed on a display device, rendered as an audio via one or more aurdio devices, or transmitted via a transmitter or output port for further processing.

FIG. 1B illustrates a workflow of the method 100 of FIG. 1A. An input video 122 of the subject is collected, for example in the manner described with reference to step 101 of FIG. 1A. The input video 122 is subjected to landmark detection for time-series data extraction 124. In this regard, some embodiments recognize that certain body parts of the subject may be better candidates for ascertaining iPPG signals for vital sign estimation. Accordingly, each frame of the video may be segmented into multiple spatial regions and each spatial region may be jointly or separately analyzed for detecting one or more landmarks corresponding to one or more body parts of the subject. Thus, each spatial region is a region of interest (ROI) for determining PPG signal.

FIG. 1C illustrates a flowchart of a method 150 for the multi-dimensional time series data extraction 124, according to some embodiments. The input video 122 is collected 152 and fragmented 154 into a plurality of frames. The individual frames of the video 122 may correspond to an image of at least one body part of the subject and as such the frames of the video may yield a sequence of images. The sequence of images may correspond to different regions of a skin of the subject, where each region in the sequence includes pixels of different intensities indicative of variation of coloration of the skin.

The time series extraction method 150 also comprises landmark detection and localization 156. The frames having a desired body part of the subject are detected for further processing. For example, in each RGB video frame the face of the subject or a part thereof may be detected. Next landmark localization is used and interpolation/extrapolation 158 of its 68-landmark output to 145 landmarks is performed. That is, to extract the ROI associated with a specific body part of the subject in an image, a plurality of landmarks locations corresponding to the specific body part of the subject is localized as part of step 156 in each image frame of the video 122. Therefore, the plurality of landmark locations may vary depending on the body part used for PPG signal determination. In an example embodiment, when the face of the person is used for determining the PPG signal, 68 landmark locations corresponding to the face of the person (i.e., 68 facial landmarks) are localized in each image frame of the video 122.

Some embodiments consider that image averaging reduces the impact of quantization noise of a camera generating the video 122, motion jitter due to imperfect landmark localization, and minor deformations due to head and face motion of the person. In response to the image averaging, the plurality of landmark locations is smoothed to extract the ROIs (e.g., the 5 facial regions). Therefore, in some embodiments, before extracting the ROI from the plurality of landmark locations, the plurality of landmark locations may be smoothed using a smoothing technique such as a moving average technique. In particular, a kernel of a predetermined size is moved over the plurality of landmark locations in the images to replace pixel values, in each landmark location, operated by the kernel, by an average value of the pixel values operated by the kernel.

For instance, 68 landmark locations may be smoothed using the moving average with a kernel of size 3-frames. The smoothed landmark locations are then used to extract the 5 ROI located around the forehead, cheeks, and chin. Thus, in each frame of the video, the average intensity of the pixels in each spatial region of the 5 spatial regions is computed. In this way, the plurality of spatial regions (or ROIs) is extracted from each image, where the plurality of spatial regions forms a sequence of images.

Referring to FIG. 1C, these landmarks are grouped 160 into small spatial areas, in each of which the mean pixel intensity of each illumination channel is computed 162. For example, in some embodiments when an RGB camera is used to acquire the video 122, the mean pixel intensity of the Red and Green channels is computed 162. In some example embodiments, instead of using multiple illumination channels, the ratio of two illumination channels may be used as the signal for further processing. For example, one or more ratios of the one of the color channel to another color channel in the video 122 may be computed, before forming the time series data. In the exemplar scenario where the video 122 is captured by an RGB camera, the ratio of the Red and Green channels may be used for further processing.

Thereafter, grouping 164 of the small spatial areas into spatial regions is performed based on the median intensity value of the areas within each spatial region of a defined cluster size. The multidimensional time series data 126 is then extracted 166 corresponding to the pixel intensities over time for each spatial region. For example, the small spatial areas may be grouped into K=5 facial regions, taking the median intensity value of the areas within each facial region. This yields a 5-dimensional time series for each video. In some embodiments, where the vital sign to be estimated is the heart rate, a Butterworth filter with cutoff frequencies [0.7, 2.5] Hz may be applied on the time series data so as to capture frequencies in a typical range of heart rates.

Referring back to FIG. 1B, each dimension of the multidimensional time series data 126 extracted at 166 may correspond to a different spatial region from the plurality of spatial regions of skin of the subject in the sequential images of the video 122. Further, each dimension may be a signal from an explicitly tracked region of interest (ROI) of the plurality of spatial regions of the skin of the subject. The tracking reduces an amount of motion-related noise. However, the multidimensional time series data 126 may still contain significant noise due to factors such as landmark localization errors, lighting variations, 3D head rotations, and deformations such as facial expressions.

The time series data 126 comprises a plurality of iPPG waveforms/signals indicative of variation of the vital signs of the subject in a time domain. Since the sequence of images are collected remotely from the subject, the iPPG signals may be subject to structured non-Gaussian noise. Thus, the multidimensional time series data 126 may be considered as a group of measured imaging PPG signals (measured iPPG signals).

Let V∈ [symbol] represent the video 122, where S is the number of video frames, C denotes the color channels, and H, W are the height and width of video frames. Also, the time series data 126 represented as Z∈ [symbol] , may be considered to have been extracted from K facial regions where the set of K time-series is assumed to capture a combination of the pulsatile signal and noise:

$$Z = {F}^{- 1} X + E . \qquad ( 1 )$$

Thus, Z∈ [symbol] is a time series of length S frames extracted from the image intensities of K facial regions of the video, F⁻¹∈ [symbol] is the oversampled inverse Fourier transform matrix, X∈ [symbol] represents the frequency coefficients of the underlying pulse signal in the K face regions, and E∈ [symbol] is a non-pulsatile noise matrix that captures noise due to multiple sources such as specular reflections, motion, and camera quantization error.

It is an objective of the deep equilibrium model-based iPPG estimation block 128 to recover the noise free iPPG signals from the measured iPPG signals that are truly reflective of the underlying pulse signal useful for vital sign estimation by denoising the multi-dimensional time series data 126. In this regard, at block 128 the objective is to recover both the pulse signal X and noise E from the signal Z. The decomposition of Z into the signal component in the Fourier domain X and the structured noise component E have to approximately satisfy the data fidelity term

$$D = \frac{1}{2} {‖ Z - A [ \begin{matrix}X \\ E\end{matrix} ] ‖}_{F}^{2} + where A = [ {F}^{- 1} I ] .$$

However, satisfying the data fidelity term alone for the decomposition is not sufficient as multiple combinations of X and E can be used to form Z. That is, not all decompositions return X and E with appropriate structure. Towards this end, deep denoisers 226b are trained to discover the appropriate structure in the Fourier domain. Instead of using explicit priors such as [symbol] _{2,1} regularization, the signal and noise priors may be encoded implicitly as a penalty function p (·,·) and its learnable scores may be employed as deep denoisers for X and E, respectively. The recovery problem is modeled as the minimization of the sum of a data fidelity and regularization term as:

$${min}_{X , E} \frac{1}{2} {‖ Z - A [ \begin{matrix}X \\ E\end{matrix} ] ‖}_{F}^{2} + λ \cdot ρ ( X , E ) , \qquad ( 2 )$$

where A=[F⁻¹ I] and λ is the scalar weight parameter for the regularizer.

The data fidelity term

$$\frac{1}{2} {‖ Z - A [ \begin{matrix}X \\ E\end{matrix} ] ‖}_{F}^{2}$$

(denoted with D) ensures consistency of the recovered signal with that of the measurements, while the regularization term p(X, E) imposes a prior. This optimization problem can be solved via the algorithmic paradigm of proximal gradient descent, which alternates between gradient descent on D and a proximal operator corresponding to the prior p(X,E).

The noise-free iPPG signals may be referred to as denoised iPPG signals 130. The denoised signals 130 are processed for vital sign estimation 132 to estimate one or more vital sign signals 134 of the subject. The process of determining the vital sign depends on the desired vital sign. For instance, if the desired vital sign is the heart rate, the Fourier transform of the denoised iPPG is first computed and the frequency at which the magnitude of the Fourier transform is highest is treated as the estimated heart rate. If the desired vital sign is the heart rate variability, peak locations in the denoised iPPG signal are determined and the time duration between the successive peaks becomes the estimate for the heart rate variability. According to some example embodiments, the vital sign may be one or a combination of pulse rate of the subject, and a heart rate variability (also referred to as “heartbeat signal”) of the subject. In some embodiments, the vital sign of the subject may be a one-dimensional signal, where the dimension is a time dimension.

The DEQ-based iPPG estimation block 128 is based on data-driven modeling of both the pulse wave as well as the structured noise in the time series data 126. The waveform recovery (for denoised signal 130) is formulated as an optimization problem in which the sum of a data fidelity term and a regularization term is to be minimized. The regularizer term is defined such that its operators are realized as operations through deep equilibrium models (DEQs). In this regard, some embodiments provide an approach wherein a deep equilibrium model is integrated as a stand-alone pseudo proximal operator in unrolling techniques, which is described later with reference to FIGS. 2A-2D. Some other embodiments provide another approach where the DEQ is integrated as the fixed-point iteration of proximal gradient descent, which is described later with reference to FIGS. 3A-3D.

FIG. 2A illustrates a block diagram of an unrolled Deep Equilibrium Model-based imaging photoplethysmography (UDEQ-iPPG) system 200 for estimating a vital sign of a subject, according to some example embodiments. The iPPG system 200 comprises a time-series extraction module 201 and a PPG estimator module 209 for generating a PPG waveform (also referred to as “PPG signal”) given a video 205 of different regions of skin of the subject as an input. The generated PPG waveform may be further processed to estimate some vital signs of the subject. Each of the time-series extraction module 201 and the PPG estimator module 209 may be embodied as a combination of hardware and software.

In some embodiments, the iPPG system 200 may optionally include an illumination source of visible wavelengths, near infrared (NIR) wavelengths or a broad spectrum with visible and NIR wavelengths. The illumination source may be configured to illuminate the skin of the subject. The iPPG system 200 may also optionally comprise a camera configured to capture a video 205 in the respective wavelengths of at least one body part of the subject (such as the face of the subject). In some example embodiments, the imaging setup comprising the illumination source and the camera may be external to the iPPG system 200. Irrespective of whether the imaging setup is part of the iPPG system 200 or not, the iPPG system 200 receives the captured video 205 of the subject as an input. The captured video 205 may correspond to the face of the subject. The video 205 includes a plurality of frames such that the video 205 contains an image of the face of the subject in each of the frames. One example of the image of the face of the subject as captured in at least one of the frames of the video 205 is shown as the image 207.

In some example embodiments, the time series extraction module 201 may partition the image 207 in each frame of the video 205 into a plurality of spatial regions 203, where the plurality of spatial regions 203 is analyzed jointly to accurately determine the PPG waveform. The partitioning (segmentation) of each image 207 is based on the realization that specific areas of the body part under consideration contain the strongest PPG signal. For example, specific areas of a face (also referred to as “regions of interest (ROIs)”) containing the strongest PPG signals are areas located around forehead, cheeks, and chin (as shown in FIG. 2A). The partitioning of each image 207 results into a sequence of images comprising different spatial regions of the plurality of spatial regions 203, where each spatial region comprises, data corresponding to at least one body part such as the skin of the subject.

The iPPG system 200 extracts a sequence of the images 207 of different regions of the skin of the subject. To that end, the iPPG system 200, for each video 205, obtains an image (for example, image 207) from each image frame of the video 205. Each image is partitioned or segmented into a plurality of spatial regions (for example, the spatial regions 203) resulting in a sequence of images corresponding to different areas of the body part (for example, the face). The partitioning of the image 207 is performed such that each spatial region comprises a specific area, of the body part, that is strongly indicative of the PPG signal. Thus, each spatial region of the plurality of spatial regions 203 is a region of interest (ROI) for determining PPG signal.

For each of the spatial region a time-series signal is derived by the time series extraction module 201. As such, the time series extraction module 201 of the iPPG system 200 transforms the sequence of images into multidimensional time series data 208. In some example embodiments, for each video 205, the time series extraction module 201 may extract a 5-dimensional time series data corresponding to pixel intensities over time of 5 facial regions (ROI), where the facial regions correspond to the plurality of spatial regions 203. In some embodiments, the multidimensional time series signal may have more or less than 5 dimensions corresponding to more or less than 5 facial regions. In some embodiments, the 5 facial regions may correspond to the right cheek, left cheek, chin, right forehead and the left forehead. The time series extraction module 201 is configured to transform the sequence of images corresponding to the plurality of spatial regions 203 into the multidimensional time series signal. To that end, pixel intensity variations of pixels from each spatial region of the plurality of spatial regions (also referred to as “different spatial regions”) 203 at an instance of time may be averaged to produce values of different dimensions of the multidimensional time-series signal for the instance of time.

In some embodiments, the time series extraction module 201 may be further configured to temporally window (or segment) the multidimensional time series signals. Accordingly, there may be a plurality of segments of the multidimensional time series signals, where at least some part of each segment of the plurality of segments overlaps with a subsequent segment of the plurality of segments forming a sequence of overlapping segments. Further, each of the segment may be normalized before submitting the multidimensional time series signals to the PPG estimator module 209. The windowed sequences may be of specific durations with a specific frame stride at interference (e.g., 10 seconds duration (250 frames at 25 fps) with a 10-frame stride at the inference), where stride indicates number of frames (e.g., 10-frames) shift over the windowed sequence (e.g., the 10 seconds windowed sequences).

The sensitivity of PPG signals to noise in measurements of intensities (e.g., pixel intensities in images) of a skin of a subject is caused at least in part by independent estimation of PPG signals from the intensities of a skin of the subject measured at different spatial positions (or spatial regions). At different locations, e.g., at different regions of the skin of the subject, the measurement intensities may be subjected to different measurement noise. When the PPG signals are independently estimated from intensities at each spatial region (e.g., the PPG signal estimated from intensities at one skin region is estimated independently of the intensities or estimated signals from other skin regions), the independence of the different estimates may cause an estimator to fail to identify such noise affecting accuracy in determining the PPG signal.

The noise includes one or more of illumination variations, pixel blurs due to motion of the person, and the like. Also, vital signs such as heartbeat are a common source of the intensity variations present in the different regions of the skin. Thus, the effect of the noise on the quality of the vital signs' estimation may be reduced when the independent estimation is replaced by a joint estimation of PPG signals measured from the intensities at different regions of the skin of the subject.

For example, within the context of performing vital sign estimation using video of face of a subject, it may be contemplated that some facial regions are physiologically known to contain better PPG signals. However, the “goodness” of these facial regions also depends on several factors such as the particular conditions in which the video was captured, facial hair of the subject, or facial occlusions and the like. Therefore, it is beneficial to identify which regions are likely to contain the most noise and remove them before any processing, so that they do not affect the signal estimates. In this regard, some example embodiments may incorporate principles and techniques aimed at computing the signal to noise ratio (SNR) for each region of the face. Some embodiments do so by throwing away a region if its SNR is below a threshold SNR or if its maximum amplitude is above a threshold amplitude. Therefore, only a select few facial regions may be chosen for further processing, thereby reducing the complexity of signal estimation.

Referring to FIG. 2A, the iPPG system 200 jointly analyzes the plurality of spatial regions 203 in order to estimate the vital sign to reduce the effect of noise. According to some example embodiments, the vital sign may be one or a combination of pulse rate of the subject, and a heart rate variability (also referred to as “heartbeat signal”) of the subject. In some embodiments, the vital sign of the subject may be a one-dimensional signal, where the dimension is a time dimension.

Some embodiments are based on the understanding that the vital sign may be estimated accurately by adopting temporal analysis. As such, the iPPG system 200 is configured to extract multidimensional time series data 208 from the sequence of images corresponding to different regions of the skin of the subject, where the multidimensional time series data 208 is used to determine the PPG signal to accurately estimate the vital sign. Some embodiments are also based on the revelation that when RGB cameras are used to acquire the video 205, in order to improve the sensitivity to illumination variations, the iPPG system 200 is configured to compute one or more ratios of the one of the color channels to another color channel in the video 205.

The estimated multi-dimensional time series data 208 is provided to the PPG estimator module 209 to recover a signal of interest (PPG signal) from the noisy multidimensional time-series data 208. The PPG estimator module 209 comprises an unrolled DEQ iPPG algorithm 209a configured to recover and output the PPG signal. The unrolled DEQ iPPG algorithm 209a is implemented by unrolling the iterations of a model-based proximal descent algorithm for the PPG estimator module 209. Each iteration contains a gradient computation step 209b that reduces a data-fitting error term and denoising steps 209c that help bring the output of the gradient step closer to a noise free PPG signal.

According to some embodiments, instead of assuming that the Fourier coefficients of the pulse signal and the noise signal are sparse, the iPPG algorithm 209 relaxes these sparsity assumptions by incorporating a learnable prior realized through deep learning networks. The unrolling based approach of the algorithm 209a imposes a learned prior on the signal and noise instead of sparsity priors, and solves for the corresponding proximal operators by “unrolling” proximal gradient descent for T iterations, where T may be a hyperparameter determined empirically.

Iteratively applying the two steps ensures that the intended result is consistent with measurements by minimizing a data fidelity term using a learned or fixed forward operator. Also, applying the signal denoiser using a learned signal prior fits the solution to a ground truth signal. Furthermore, in order to denoise the time series data 208 the algorithm 209a repeatedly applies the DEQ based denoising operator 209c until the output converges. Expanding upon these understandings, some embodiments consider that the frequency coefficients of the underlying heart rate signal can be obtained as a fixed point of the denoising operator. The choice of DEQ based denoiser decouples the choice of solver from the computation of the endpoint or root, and can backpropagate through this point analytically and independently of the forward pass. Also, DEQs can simultaneously perform forward inference as well as input optimization on problems such as gradient-based meta-learning.

The algorithm 209a is based on the recognition that the structure of the iPPG signals can be learned and/or represented as a deep equilibrium model that directly finds via root-finding techniques, equilibrium points to which the frequencies of the iPPG signals converge. The algorithm 209a solves a structured recovery problem with a regularizer enforcing a neural network discovered structure on the iPPG signals. Recalling from eqn. (2), the structured recovery problem can be modeled as the minimization of the sum of the data fidelity and regularization term. At each unrolling iteration of the algorithm 209, X can be denoised to a fixed-point and a deep equilibrium denoiser can be used for denoising.

The number of iterations of the algorithm 209a may be a dynamic variable defined by an operator or may be defined in relation to the vital sign being estimated. In some example embodiments, an input may be provided to run the algorithm 209a for the defined number of iterations. In some other example embodiments, the threshold for the number of iterations may be fetched from a database defining the number of iterations in relation to one or more of the vital sign to be estimated, the body part considered for imaging, gender, age, or other physiological properties of the subject. In some example embodiments, the threshold for the number of iterations may be a hyperparameter of the unrolled DEQ iPPG algorithm 209a. The value corresponding to the number of iterations may serve as a termination condition for the algorithm 209a. According to some embodiments, the unrolled DEQ-iPPG algorithm 209a estimates the iPPG signal in frequency domain.

Using the recovered PPG signals after a certain number of iterations of the unrolled DEQ iPPG algorithm 209a, some vital signs 211 of the subject may be estimated. For example, the estimate of a vital sign in the time window may be considered as the frequency component for which the power of the frequency spectrum is maximum.

FIG. 2B illustrates a flowchart of some steps of a method 220 executed by the iPPG system 200 for estimating a vital sign 211 of a subject, according to some example embodiments. FIG. 2B is described in conjunction with FIG. 2A. The video 205 of the subject is received by the iPPG system 200 and time series sequence of iPPG signals 208 are collected 222. The time series data 208 may be collected in accordance with the workflow defined with respect to FIGS. 1B and 1C. The collected iPPG signals are modeled to be a combination of an underlying pulse signal and a non-pulsatile noise. Coefficients of the underlying pulse signal (X) and the non-pulsatile noise matrix (E) are initialized 224. The iPPG algorithm 209a then iteratively performs 226 gradient descent steps

$$\frac{\partial Z}{\partial {X}^{*}} , \frac{\partial Z}{\partial {E}^{*}} ,$$

followed by forward operations through neural networks [symbol] (·) and [symbol] (·), respectively for T iterations.

For each input time window containing S frames, the time series data 208 containing the average intensity of each of K face regions in every frame is extracted. Stacking these signals into a matrix Z of size S×K, it is assumed that that these region-specific signals share a quasi-periodic pulse signal that admits a structured representation in the Fourier domain. Therefore, from eqn. (1), the observation of the vital sign i.e., the measured iPPG signal can be modeled as:

$$Z = Y + E = {F}^{- 1} X + E , \qquad ( 3 )$$

where F⁻¹ is the oversampled inverse Fourier Transform matrix of size S×N. In eqn. (3) Y of size S×K represents the pulsatile signal in all K regions in the time domain and X of size N×K represents the pulsatile signal in all K regions in the frequency domain. E is a real matrix of S×K that represents the structured noise component that captures the non-pulse-related fluctuations in the iPPG signals Z. Here, Z is measured in the time domain, and processed in the Fourier domain using the Fourier transform F. This is based on the realization that the Fourier domain acts as a structure enforcer on the estimated PPG signal Y in the Fourier domain where X, which is given Fourier transform on Y has a simpler structure than in the measurement domain. X is the signal processed in the Unrolled DEQ iPPG algorithm 209a, instead of Y. On the other hand, the structured noise component E is procesed by the Unrolled iPPG algorithm 209a in the time domain. That is, X and E, which are the components of Z are processed in different domains. In this example embodiment, these two domains are Fourier domain and time domain respectively. In other embodiments, these domains can also be the wavelet domain or an appropriate dictionary learned from data.

The Unrolled DEQ iPPG algorithm 209a unrolls proximal gradient descent for Eq. (2) for T iterations. In each iteration, gradient updates 226a are performed on X and E followed by forward propagation through the learned signal denoiser R 226b and the denoiser for the structured noise component Q 226c.

FIG. 2C illustrates two generic iterations of the Unrolled DEQ-iPPG (UDEQ-iPPG) algorithm 209a, according to some example embodiments. Given a step size a, the updates on X and E are given by

$$[ \begin{matrix}{\tilde{X}}_{t + 1} \\ {\tilde{E}}_{t + 1}\end{matrix} ] = [ \begin{matrix}{X}_{t} \\ {E}_{t}\end{matrix} ] - α {∇}_{[ \begin{matrix}X \\ E\end{matrix} ]} D ( [ \begin{matrix}{X}_{t} \\ {E}_{t}\end{matrix} ] ) , \qquad ( 4 )$$

$${X}_{t + 1} = R ( {\tilde{X}}_{t + 1} ) ,$$

$${E}_{t + 1} = Q ( {\tilde{E}}_{t + 1} ) .$$

where Xₜ₊₁ corresponds to the update on Xₜ and Eₜ₊₁ corresponds to the update on Eₜ.

Referring to FIG. 2C, instantiating the learnable function [symbol] (·) as a DEQ operator 226b, gradient descent and denoising then become

$$( a ) {[ \begin{matrix}{\tilde{X}}_{t + 1} \\ {\tilde{E}}_{t + 1}\end{matrix} ]}_{( a )} = [ \begin{matrix}{X}_{t} \\ {E}_{t}\end{matrix} ] - α {∇}_{[ \begin{matrix}X \\ E\end{matrix} ]} D ( [ \begin{matrix}{X}_{t} \\ {E}_{t}\end{matrix} ] ) , \qquad ( 5 )$$

$$( ) α {A}^{T} Z + ( I - α {A}^{T} A ) [ \begin{matrix}{X}_{t} \\ {E}_{t}\end{matrix} ] ,$$

$$( b ) {X}^{*} = ( {X}^{*} ; {\tilde{X}}_{t + 1} )$$

$${X}_{t + 1} = {X}^{*} ,$$

$${E}_{t + 1} = {{θ}_{Q}}_{} ( {\tilde{E}}_{t + 1} )$$

where equations labeled “(a)” above the equals sign correspond to gradient steps and “(b)” correspond to proximal operators.

The DEQ operator of 209c applies an implicit model for solving a non-linear system prescribed by a criterion such as the root of an equation, and backpropagates through this root/endpoint independent of the forward pass. The DEQ operator utilizes a Deep Equilibrium Model (DEQ) that approximate “infinitely deep” models by solving for the fixed-point, or equilibrium point z*, of a function z*=ƒ_{θ}(z*, x) where x is the input. The choice of solver for z*—whether it be Broyden's Method, Peaceman-Rachford splitting method, Anderson Acceleration for fixed point iterations, or any other suitable method—is independent of the backpropagation procedure. Once this point is found, the Implicit Function Theorem (IFT) may be used to compute the gradients of the parameters. Details of the IFT are provided later in the disclosure.

FIG. 2D illustrates a block diagram of a DEQ operator 251 utilized in each iteration of the UDEQ-iPPG algorithm of FIG. 2C, according to some example embodiments. The choice of solver can be Broyden's Method, Peaceman-Rachford splitting method, Anderson Acceleration for fixed point iterations, or any other suitable method. The solver 253 takes as input a gradient updated signal \tilde{X}ₜ as well as an initial estimate of the fixed point X*^{[0]} and runs the iterations of the solver until a fixed point X* is found, where the solver outputs X* when the input to the DEQ operator is also X*. According to some embodiments, one possible initialization of X*^{[0]} can be a matrix of all zeros.

Referring back to FIG. 2B, the noise component E, however, is an unstructured signal for which there is no corresponding fixed-point. Therefore, the denoiser Q(·) is instantiated as a feed-forward neural network 226c without the fixedpoint constraints of DEQs.

The UDEQ-iPPG algorithm 209a is unrolled for T iterations to yield 228 the frequency coefficients corresponding to each iPPG signal. At step 230, the method 220 sums the frequency coefficients across individual bins for all iPPG signals and the bin with the highest frequency is selected 232 as the vital sign signal. In some embodiments, to find the vital sign, power in every frequency bin across all K regions may be summed, and the frequency with the maximum power is selected to be the frequency of the signal representing the vital sign. For example, in one embodiment an estimated vital sign is the frequency of the heartbeat of the subject over the period of time.

FIG. 3A illustrates a block diagram of a Deep Equilibrium Proximal Gradient Descent (DE-Prox) based imaging photoplethysmography (DE-Prox-iPPG) system 300 for estimating a vital sign of a subject, according to some example embodiments. FIG. 3B illustrates a flowchart of some steps performed by the DE-Prox-iPPG system 300, and will be described in conjunction with FIG. 3A. The iPPG system 300 comprises a time-series extraction module 301 and a PPG estimator module 309 for generating a PPG waveform (also referred to as “PPG signal”) given a video 305 of different regions of skin of the subject as an input. The generated PPG waveform may be further processed to estimate some vital signs of the subject. Each of the time-series extraction module 301 and the PPG estimator module 309 may be embodied as a combination of hardware and software. But for the PPG estimator module 309, other components and properties of the iPPG system 300 remain the same as that of the iPPG system 200 of FIG. 2A.

In a manner similar to the one described previously with respect to FIG. 2A, the time series extraction module 301 extracts time series data 308 corresponding to each spatial region 303 of the sequential image 307 corresponding to each frame of the video 305. The estimated multi-dimensional time series data 308 is provided to the PPG estimator module 309 as a time series sequence of iPPG signals that are collected 322 to recover a signal of interest (PPG signal) from the noisy multidimensional time series data 308. Referring to FIG. 3A, the PPG estimator module 309 implements a proximal gradient descent based algorithm 309a that integrates a DEQ as a fixed point iteration of proximal gradient descent approach. The algorithm 309a, also referred to as Deep Equilibrium Proximal Gradient Descent iPPG algorithm (DE-Prox iPPG), recovers and outputs the PPG signals corresponding to the time series data 308. In this regard, the DE-Prox iPPG algorithm 309a encapsulates the entire iteration map (i.e., gradient descent followed by the pseudo-proximal operations) in a DEQ, as shown in FIG. 3C, and iterates these steps until a fixed-point is found for the optimization variables. Thus, each iteration contains a gradient computation step 309b that reduces a data-fitting error term and denoising steps 309c that help bring the output of the gradient step closer to a noise free PPG signal.

It is a recognition of the DE Prox iPPG algorithm 309a that a fixed point for the frequency coefficients X* is inherently connected to a fixed-point of the noise E*. Accordingly, as shown in FIG. 3B, the coefficients of the underlying pulse signal X* and the non-pulsatile noise matrix E* are concatenated 324 and the algorithm 309a optimizes for the fixed-point of the concatenation of the two variables, [X*, E*]^{T}. A deep equilibrium model ƒ_{θ}([X*, E*]^{T}, Z) is defined which iterates through gradient descent steps 326a and pseudo-proximal operators 326b, 326c until convergence as: (a)

$$( ) ⋆ {f}_{θ} ( {[ {X}^{*} , {E}^{*} ]}^{T} , Z ) \qquad ( 5 )$$

$$( b ) [ \begin{matrix}\tilde{{X}^{*}} \\ {\tilde{E}}^{*}\end{matrix} ] = [ \begin{matrix}{X}^{⋆} \\ {E}^{⋆}\end{matrix} ] - α {∇}_{[ \begin{matrix}{X}^{⋆} \\ {E}^{⋆}\end{matrix} ]} D ( [ \begin{matrix}{X}^{⋆} \\ {E}^{⋆}\end{matrix} ] ) ,$$

$${X}^{*} = ( {\tilde{X}}^{*} ) ,$$

$${E}^{*} = {{θ}_{Q}}_{} ( {\tilde{E}}^{*} ) ,$$

where equations labeled “(a)” above the equals sign correspond to gradient steps and “(b)” correspond to pseudo-proximal operators.

It may be noted that DE-Prox-iPPG, as compared to UDEQ-iPPG, computes the gradient and denoising (pseudo-proximal) steps for a variable number of iterations, i.e., until a fixed point of the global system is found. While UDEQ-iPPG 209a unrolls for T iterations, the number of iterations in DE-Prox-iPPG 309a varies depending on one or more characteristics of the input signal such as its noise level.

FIG. 3C illustrates the iterative DE-Prox-iPPG algorithm executed by the system of FIG. 3A, according to some example embodiments. The time series data Z (extracted from the sequential images 307 of input video) and the two concatenated optimization variables [X*, E*]^{T} are passed through the block 309b which performs gradient descent steps

$$\frac{\partial Z}{\partial {X}^{*}} , \frac{\partial Z}{\partial {E}^{*}}$$

to obtain gradient updates that are then passed through denoising operators [symbol] and [symbol] . This process is repeated until fixed-point convergence is achieved according to the condition ƒ_{θ}([X*, E+]^{T}; Z)=[X*, E*]^{T}.

Referring back to FIG. 3B, the iterations until convergence yield 328 the frequency coefficients corresponding to each iPPG signal. At step 330, the frequency coefficients across individual bins for all iPPG signals are summed and the bin with the highest frequency is selected 332 as the vital sign signal. In some embodiments, to find the vital sign, power in every frequency bin across all K regions may be summed, and the frequency with the maximum power is selected to be the frequency of the signal representing the vital sign. For example, in one embodiment an estimated vital sign is the frequency of the heartbeat of the subject over the period of time.

FIG. 3D illustrates an architecture of denoisers [symbol] and [symbol] utilized by the DE-Prox-iPPG system 300 of FIG. 3A, according to some example embodiments. In the DE-Prox-iPPG architecture of the iPPG system 300, one or more encoder-decoder denoiser architecture is applied to the output of the gradient step 309b in each iteration. Thus, the PPG estimator module 309 comprises one or more time series denoiser neural networks (also referred to as “denoisers”) 309c. As is shown in FIG. 3D, the encoder 352 for the network [symbol] is realised through the blocks 352a and 352b, the decoder for the network [symbol] is realised through the blocks 354a and 354b, while the encoder for the network [symbol] is realised through the blocks 356a and 356b and the decoder for the network [symbol] is realised through the blocks 358a and 358b. As the network [symbol] operates on Fourier coefficients Xₜ, its network weights are complex-valued (blocks shown in bold thick bounds), while the network weights for Q, which operates on Eₜ, are real-valued (blocks shown in thin bounds). The networks consist of two downsampling convolutional blocks 352a and 352b for network R and 356a and 356b for network Q, with a stride of 2, in which the number of channels is increased from 5 to 32 and then from 32 to 64. The two upsampling blocks 354a and 354b for network R and 358a and 358b for network Q, are implemented using transposed convolution with a stride of 2, decreasing the number of channels from 64 to 32 and from 32 to 5. Each convolutional layer has a kernel size of 16 in the temporal dimension and is followed by a ReLU nonlinearity and then a batch normalization layer.

In some embodiments, during training, parameters of the neural networks R and Q are updated so as to minimize the mean squared error loss between the output signal after T unrolled iterations, Y^{T}=F⁻¹X^{T}, and the ground-truth waveform Z_{gt}. Minibatch stochastic gradient descent using backpropagation may be used to update the parameters of the denoisers R and Q.

In order to find the fixed-point, whether it be in UDEQ-iPPG or DE-Prox-iPPG, the iPPG system 200/300 find the root of g ([X*, E*]^{T}; Z)=X*−ƒ_{θ}([X*, E*]^{T}; Z)=0. This technique permits efficient root-solving algorithms such as Broyden's Method or Anderson Acceleration to solve for the fixed-point. During training, the iPPG system iterates for T iterations for UDEQ-iPPG 209a, or until fixed-point convergence for DE-Prox-iPPG 309a, as shown in FIGS. 2C and 3C, after which both models' parameters must be updated after computing some loss [symbol] . While θ_{Q} (denoting the parameters of [symbol] ) in UDEQ-iPPG can be updated via the standard Autograd module, updating the parameters [symbol] in UDEQ-iPPG and both [symbol] , θ_{Q} in DE-Prox-iPPG requires computing gradients with respect to the parameters. One approach in this regard is to backpropagate through iterations of the solver, while another approach—a memory efficient backpropagation scheme uses the Implicit Function Theorem, shown below.

Implicit Function Theorem (IFT): Given the fixed-point flow representation z*=z*=[X*, E*]^{T} and the corresponding loss [symbol] (X*, X_{gt}), the gradient of DEQ flow is given by

$$\frac{\partial ℒ}{\partial θ} = \frac{\partial ℒ}{\partial {z}^{*}} {( I - \frac{\partial {f}_{θ} ( {z}^{*} )}{\partial {z}^{*}} )}^{- 1} \frac{\partial {f}_{θ} ( {z}^{*} , Z )}{\partial θ} . \qquad ( 6 )$$

The Mean Squared Error Loss between the final output Z_{final}=F⁻¹X_{final} and the ground-truth PPG signal Z_{gt}. Here, X_{final} is X_{T} in UDEQ-iPPG and is X*, the fixed-point, in DE-Prox-iPPG. Some embodiments penalize the Frobenius norm of the Jacobian with respect to the fixed-point z*, which is given by

$${J}_{{f}_{θ}} = \frac{\partial {f}_{θ} ( {z}^{*} )}{\partial {z}^{*}} ,$$

where J_{ƒ}_{_{θ}}∈ [symbol] . The Hutchinson estimator may be used to estimate ∥J_{ƒ}_{_{θ}}∥₂² and compute

$$tr ( {J}_{{f}_{θ}} {J}_{{f}_{θ}}^{T} ) = {𝔼}_{ϵ ∈ 𝒩 ( , I , d )} [ {‖ {ϵ}^{T} {J}_{{f}_{θ}} ‖}_{2}^{2} ] \qquad ( 7 )$$

The final loss is composed of the mean squared error (MSE) between the recovered waveform and the ground truth waveform, modulated by the Jacobian regularization penalty:

$$ℒ = {ℒ}_{m s e} ( {Z}_{final} , {Z}_{g t} ) + λ \frac{{‖ {ϵ}^{T} {J}_{{f}_{θ}} ( {z}^{*} ) ‖}_{2}^{2}}{d} , \qquad ( 8 )$$

where λ determines the strength of regularization. To determine the heart-rate, the multi-channel recovered frequency coefficients X_{final} are summed across each bin, after which the bin with the maximum power is the vital sign estimate.

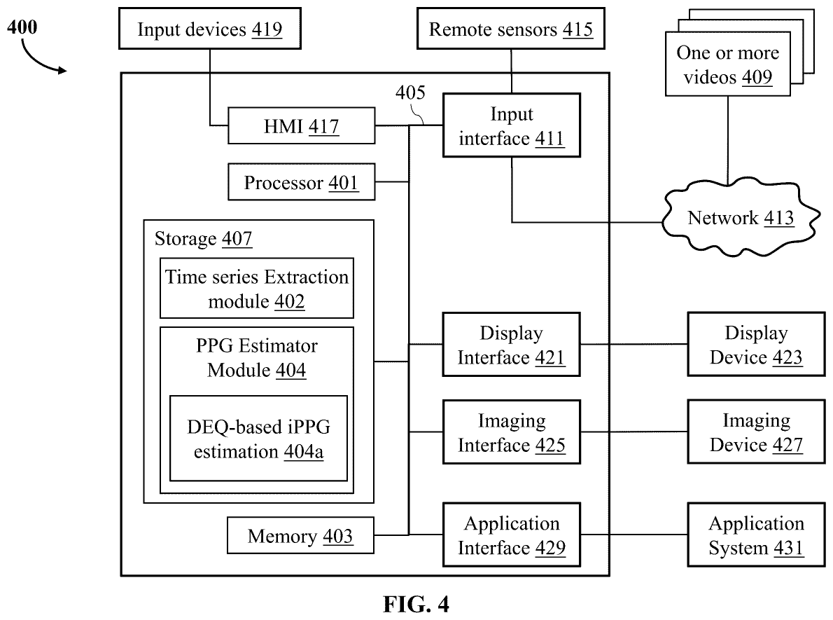

FIG. 4 illustrates a block diagram of an iPPG system 400, according to an example embodiment. The system 400 includes a processor 401 configured to execute stored instructions, as well as a memory 403 that stores instructions that are executable by the processor 401. The processor 401 may be a single core processor, a multi-core processor, a computing cluster, or any number of other configurations. The memory 403 can include random access memory (RAM), read only memory (ROM), flash memory, or any other suitable memory systems. The processor 401 is connected through a bus 405 to one or more input and output devices.

The instructions stored in the memory 403 correspond to an iPPG method for estimating the vital signs of the person based on a set of iPPG signals' waveforms measured from different regions of a skin of a subject. The iPPG system 400 may also include a storage device 407 configured to store various modules such as the time-series extraction module 402 and the PPG estimator module 404, where the PPG estimator module 404 comprises one or more of the implemented iPPG algorithms 209a or 309a. The aforesaid modules stored in the storage device 407 are executed by the processor 401 to perform the vital signs estimations. The vital sign may correspond to a pulse rate of the person or heart rate variability of the person. The storage device 407 may be implemented using a hard drive, an optical drive, a thumb drive, an array of drives, or any combinations thereof.

The time-series extraction module 402 obtains an image in each frame of a video of one or more videos 409 that is fed to the iPPG system 400, where the one or more videos 409 comprises a video of a body part of a subject whose vital signs are to be estimated. The one or more videos may be recorded by one or more suitable imaging devices. The time-series extraction module 402 may partition the image from each frame into a plurality of spatial regions corresponding to ROI of the body part that are strong indicators of PPG signal, where the partitioning of the image into the plurality of spatial regions form a sequence of images of the body part. Each image comprises different region of a skin of the body part in the image. The sequence of images may be transformed into a multidimensional time-series signal in the manner described previously with reference to FIG. 1C. The multidimensional time-series signal may be provided to the PPG estimator module 404. The PPG estimator module 404 may utilize one of the iPPG algorithms 209a or 309a to process the multidimensional time-series signal for estimating the PPG waveform, where the PPG waveform is used to estimate the vital signs of the subject.

The iPPG system 400 includes an input interface 411 such as an input port to receive the one or more videos 409. For example, the input interface 411 may be a network interface controller adapted to connect the iPPG system 400 through the bus 405 to a network 413.

Additionally, or alternatively, in some implementations, the iPPG system 400 is connected to a remote sensor 415, such as an imaging sensor, to collect the one or more videos 409. In some implementations, a human machine interface (HMI) 417 within the iPPG system 400 connects the iPPG system 400 to input devices 419, such as a keyboard, a mouse, trackball, touchpad, joystick, pointing stick, stylus, touchscreen, and among others to receive inputs from a user or operator.

The iPPG system 400 may be linked through the bus 405 to an output interface to render the PPG waveform. For example, the iPPG system 400 may include a display interface 421 adapted to connect the iPPG system 400 to a display device 423, wherein the display device 423 may include, but not limited to, a computer monitor, a projector, or mobile device. The iPPG system 400 may also include and/or be connected to an imaging interface 425 adapted to connect the iPPG system 400 to an imaging device 427.

In some embodiments, the iPPG system 400 may be connected to an application interface 429 through the bus 405 adapted to connect the iPPG system 400 to an application system 431 that can be operated based on the estimated vital signals. In an exemplary scenario, the application system 431 may be a patient monitoring system, which uses the vital signs of a patient. In another exemplary scenario, the application system 431 is a driver monitoring system, which uses the vital signs of a driver to determine if the driver can drive safely, e.g., whether the driver is drowsy or not.

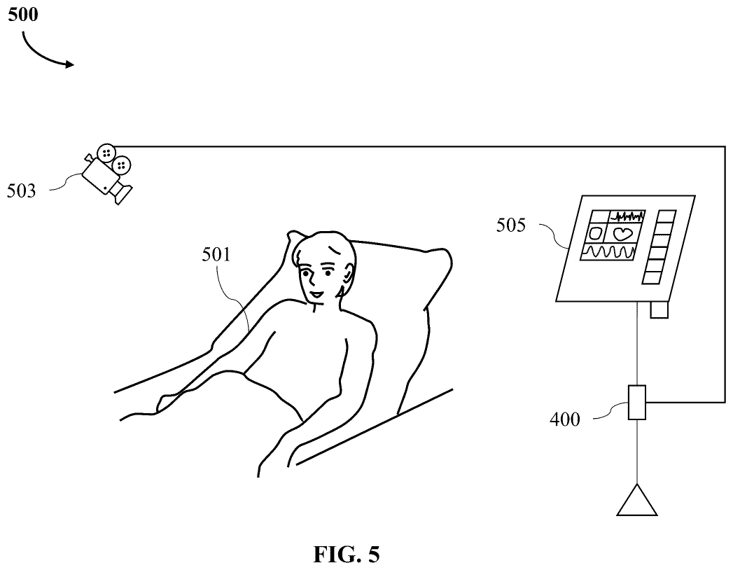

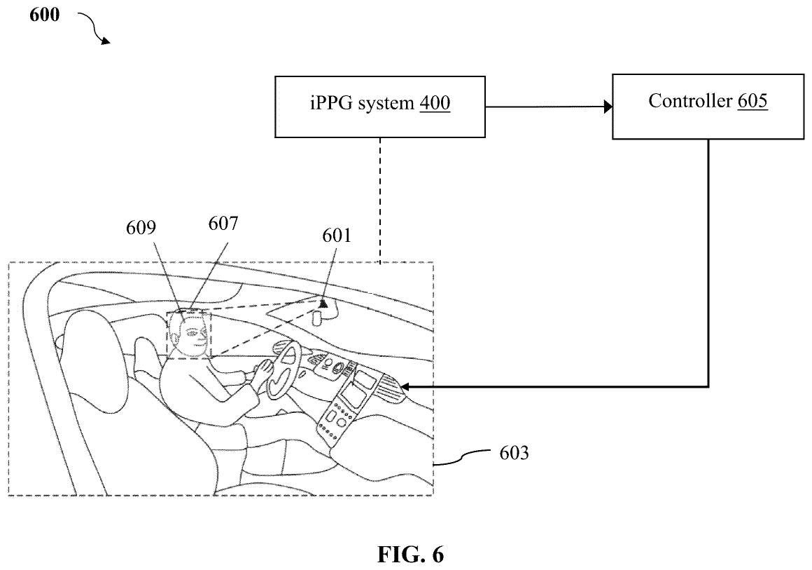

The proposed approaches for estimating vital signs of subjects may be used for several control applications, some of which are described next with reference to FIGS. 5 and 6.

In some example embodiments, the subject whose vital signs are to be estimated may be a patient. In such example scenarios, the iPPG system may be used for monitoring the vital signs of the patient. FIG. 5 illustrates an example patient monitoring system 500 using the iPPG system 400, according to an example embodiment. To monitor vital signs of the patient 501, a camera 503 is used to image the patient 501 to obtain a video sequence of the patient 501. The camera 503 may include a CCD or CMOS sensor for converting incident light and the intensity variations thereof into an electrical signal. The camera 503, particularly non-invasively, captures light reflected from a skin portion of the patient 501. A skin portion may thereby particularly refer to the forehead, neck, wrist, part of the arm, or some other portion of the patient's skin. A light source, e.g., a near-infrared light source, may be used to illuminate the patient or a region of interest including a skin portion of the patient.

Based on the captured images, the iPPG system 400 determines the vital signs of the patient 501 according to the example embodiments described previously. In particular, the iPPG system 400 determines the vital signs such as the heart rate, the breathing rate or the blood oxygenation of the patient 501. Further, the determined vital signs may be displayed on an operator interface 505 for presenting the determined vital signs. Such an operator interface 505 may be a patient bedside monitor or may also be a remote monitoring station in a dedicated room in a hospital or even in a remote location in telemedicine applications.

In some example embodiments, the subject whose vital signs are to be estimated may be a driver or a passenger in a vehicle. FIG. 6 illustrates a passenger assistance system 600 using the iPPG system 400, according to an example embodiment. An illumination source such as a NIR light source and/or a NIR camera 601 is arranged within a vehicle 603. In particular, the NIR camera 601 may be arranged in a field of view (FOV) 607 capturing the driver or passenger 605. The iPPG system 400 is integrated into the vehicle 603. The NIR light source is configured to illuminate skin of an occupant of the vehicle such as the driver or passenger 605, and the NIR camera 601 is configured to record the video of the driver or passenger 605 in real time. Further, the NIR videos are fed to the iPPG system 400 to measure the iPPG signals from different regions of the skin of the driver or passenger 605. The iPPG system 400 receives the measured iPPG signals and determines the vital sign, such as pulse rate, of the driver or passenger 605.

Having obtained the vital sign of the passenger or driver 605, the iPPG system 400 may process the estimated vital sign to check for a condition of the passenger or driver 605. In some example embodiments, the processing of the estimated vital sign of the passenger or driver 605 may be performed by a control system of the vehicle 603 or a remote server communicatively coupled with the control system of the vehicle 603. For example, an estimated vital sign of the passenger or driver 605 may be compared with a permissible threshold to ascertain a medical fitness of the passenger or driver 605. If the result of comparison indicates that the medical fitness of the passenger or driver 605 is not suitable, an appropriate action may be initiated. For example, the processor of iPPG system 400 may produce one or more control action commands, based on the estimated vital signs of the driver 605 of the vehicle 603. The one or more control action commands may include vehicle braking, steering control, generation of an alert notification, initiation of an emergency service request, or switching of a driving mode from manual to automatic or vice versa. The one or more control action commands may be transmitted to the controller 605 of the vehicle 603. The controller 605 may control the vehicle 603 according to the one or more control action commands. For example, if the determined pulse rate of the driver is very low, then the driver 605 may be experiencing a heart attack. Consequently, the iPPG system 400 may produce control commands for reducing a speed of the vehicle and/or steering control (e.g., to steer the vehicle to a shoulder of a highway and make it come to a halt) and/or initiate an emergency service request.

In this way, several example embodiments described herein may be used in real world applications for medical diagnosis and well being, vehicle assistance, and patient monitoring.

The above description provides exemplary embodiments only, and is not intended to limit the scope, applicability, or configuration of the disclosure. Rather, the above description of the exemplary embodiments will provide those skilled in the art with an enabling description for implementing one or more exemplary embodiments. Contemplated are various changes that may be made in the function and arrangement of elements without departing from the spirit and scope of the subject matter disclosed as set forth in the appended claims.

Specific details are given in the above description to provide a thorough understanding of the embodiments. However, understood by one of ordinary skill in the art can be that the embodiments may be practiced without these specific details. For example, systems, processes, and other elements in the subject matter disclosed may be shown as components in block diagram form in order not to obscure the embodiments in unnecessary detail. In other instances, well-known processes, structures, and techniques may be shown without unnecessary detail in order to avoid obscuring the embodiments. Further, like reference numbers and designations in the various drawings indicated like elements.

Also, individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed but may have additional steps not discussed or included in a figure. Furthermore, not all operations in any particularly described process may occur in all embodiments. A process may correspond to a method, a function, a procedure, a subroutine, a subprogram, etc. When a process corresponds to a function, the function's termination can correspond to a return of the function to the calling function or the main function.

Furthermore, embodiments of the subject matter disclosed may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

Various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine. Typically, the functionality of the program modules may be combined or distributed as desired in various embodiments.

Embodiments of the present disclosure may be embodied as a method, of which an example has been provided. The acts performed as part of the method may be ordered in any suitable way. Accordingly, embodiments may be constructed in which acts are performed in an order different than illustrated, which may include performing some acts concurrently, even though shown as sequential acts in illustrative embodiments. Although the present disclosure has been described with reference to certain preferred embodiments, it is to be understood that various other adaptations and modifications can be made within the spirit and scope of the present disclosure. Therefore, it is the aspect of the append claims to cover all such variations and modifications as come within the true spirit and scope of the present disclosure.

1. A remote photoplethysmography (RPPG) system for estimating a vital sign signal of a subject, comprising:
a memory configured to store executable instructions; and
a processor coupled with the memory, wherein the stored instructions, when executed by the processor, cause the RPPG system to:
collect a sequence of images of different regions of skin of the subject, each region including pixels of different intensities indicative of variation of coloration of the skin;
transform the sequence of images into a sequence of imaging photoplethysmography (iPPG) signals indicative of variation of the vital signs of the subject in a time domain, wherein the iPPG signals are subject to sparse non-Gaussian noise;
denoise the iPPG signals by solving a structured recovery problem with a regularizer enforcing a neural network discovered structure on the iPPG signals; and
output the vital sign signal corresponding to the denoised iPPG signals.

2. The RPPG system of claim 1, wherein the processor is configured to solve the structured recovery problem using unrolled gradient descent, wherein the regularizer is integrated as a fixed-point iteration of the unrolled gradient descent.

3. The RPPG system of claim 1, wherein the processor is configured to solve the structured recovery problem using unrolled gradient descent, wherein the regularizer is integrated as a standalone pseudo-proximal operator denoising interim outputs of the structured recovery between different iterations of the unrolled gradient descent.

4. The RPPG system of claim 1, wherein the regularizer includes a learned regularization term implemented using a deep equilibrium model (DEQ), and wherein the processor is configured to solve the structured recovery problem using unrolled gradient descent, with a predetermined number of iterations, wherein for each iteration of the predetermined number of iterations, the DEQ is executed multiple times to a fixed-point of interim outputs of the unrolled gradient descent.

5. The RPPG system of claim 3, wherein the iPPG signals comprise a pulsatile signal and a noise signal, wherein the unrolled gradient descent unrolls iterations of a learned proximal gradient descent algorithm with a learned prior for each of the pulsatile signal and the noise signal, and wherein the proximal gradient descent algorithm comprises a feed-forward pass through a deep neural network.

6. The RPPG system of claim 1, wherein the processor is further configured to estimate the vital sign signal by minimizing a difference between the sequence of iPPG signals and the denoised iPPG signals using a gradient descent minimization.

7. The RPPG system of claim 1, wherein the processor is further configured to estimate the vital sign signal by minimizing a difference between the sequence of iPPG signals and the denoised iPPG signals using a proximal gradient descent minimization.

8. The RPPG system of claim 1, further comprising a controller communicatively coupled to a machine and the processor, wherein the controller is configured to:
receive the vital sign signal of the subject; and
generate one or more control commands for controlling the machine, based on the received vital sign signal of the subject.

9. The RPPG system of claim 1, wherein the processor is further configured to:
estimate noise in the sequence of iPPG signals; and
modify the denoised iPPG signals with the estimated noise for minimizing a difference between the sequence of iPPG signals and the denoised iPPG signals.

10. The RPPG system of claim 9, wherein the noise is processed with a noise neural network to enforce an implicit structure on the noise and generate a structured component of the noise.

11. The RPPG system of claim 10, wherein the noise neural network is trained with ground truth iPPG signals measured using contact sensing.

12. A computer-implemented method for estimating a vital sign signal of a subject, comprising:
collecting a sequence of images of different regions of skin of the subject, each region including pixels of different intensities indicative of variation of coloration of the skin;
transforming the sequence of images into a sequence of imaging photoplethysmography (iPPG) signals indicative of variation of the vital signs of the subject in a time domain, wherein the iPPG signals are subject to sparse non-Gaussian noise;
denoising the iPPG signals by solving a structured recovery problem with a regularizer enforcing a neural network discovered structure on the iPPG signals; and
outputting the vital sign signal corresponding to the denoised iPPG signals.

13. The method of claim 12, further comprising solving the structured recovery problem using unrolled gradient descent, wherein the regularizer is integrated as a fixed-point iteration of the unrolled gradient descent.

14. The method of claim 12, further comprising solving the structured recovery problem using unrolled gradient descent, wherein the regularizer is integrated as a standalone pseudo-proximal operator denoising interim outputs of the structured recovery between different iterations of the unrolled gradient descent.

15. The method of claim 12, wherein the regularizer includes a learned regularization term implemented using a deep equilibrium model (DEQ), and wherein denoising the iPPG signals comprises solving the structured recovery problem using unrolled gradient descent with a predetermined number of iterations, wherein for each iteration of the predetermined number of iterations, the DEQ is executed multiple times to a fixed-point of interim outputs of the unrolled gradient descent.

16. The method of claim 12, further comprising estimating the vital sign signal by minimizing a difference between the sequence of iPPG signals and the denoised iPPG signals using a gradient descent minimization.

17. The method of claim 12, further comprising estimating the vital sign signal by minimizing a difference between the sequence of iPPG signals and the denoised iPPG signals using a proximal gradient descent minimization.

18. The method of claim 12, further comprising generating one or more control commands for controlling the machine, based on the vital sign signal of the subject.

19. The method of claim 12, further comprising:
estimating noise in the sequence of iPPG signals; and
modifying the denoised iPPG signals with the estimated noise for minimizing a difference between the sequence of iPPG signals and the denoised iPPG signals.

20. A non-transitory computer readable medium having stored thereon computer-executable instructions which when executed by a computer, cause the computer to perform a method for estimating a vital sign signal of a subject, the method comprising:
collecting a sequence of images of different regions of skin of the subject, each region including pixels of different intensities indicative of variation of coloration of the skin;
transforming the sequence of images into a sequence of imaging photoplethysmography (iPPG) signals indicative of variation of the vital signs of the subject in a time domain, wherein the iPPG signals are subject to sparse non-Gaussian noise;
denoising the iPPG signals by solving a structured recovery problem with a regularizer enforcing a neural network discovered structure on the iPPG signals; and
outputting the vital sign signal corresponding to the denoised iPPG signals.
